(19) United States
(12) Patent Application Publication
(10) Pub. No.: US 2025/0186063 A1
(43) Pub. Date: Jun. 12, 2025

(54) OSSEODENSIFICATION BUR WITH TIP RELIEF AND METHOD OF USE

(71) Applicant: Huwais IP Holding LLC, Jackson, MI (US)

(72) Inventor: Salah HUWAIS, Jackson, MI (US)

(21) Appl. No.: 18/976,969

(22) Filed: Dec. 11, 2024

Related U.S. Application Data
(63) Continuation of application No. PCT/US24/59293, filed on Dec. 10, 2024.
(60) Provisional application No. 63/608,920, filed on Dec. 12, 2023.

(51) Int. Cl. A61B 17/16

(52) U.S. Cl. CPC A61B 17/1615

(57) ABSTRACT

A rotary tool configured for high-speed condensing and/or cutting action to enlarge a hole. The tool has a body around which is formed a plurality of flutes. Each flute has a cutting face on one side and a densifying face on the other side. A land between adjacent flutes establishes a substantially margin-less working edge along each cutting face. The working edges are configured to produce osseodensification when the tool is operated in the condensing mode. A transverse valley is formed at the apical end of the body. The valley is a relief in the tip that creates several secondary cut/grind edges that are useful to produce and evenly distribute bone debris. When withdrawing the rotary osteotome from an enlarged hole, a small button of bone debris remains at the bottom of the hole. The button remnant facilitates new bone ingrowth and osseointegration of a subsequently installed anchor.

### CROSS REFERENCE TO RELATED APPLICATIONS

This application is a continuation of International Patent Application No. PCT/US24/59293 filed on Dec. 10, 2024 which claims priority to U.S Provisional Patent Application US 63/608,920 filed on Dec. 12, 2023, the entire disclosures of which are hereby incorporated by reference and relied upon.

### BACKGROUND OF THE INVENTION

### Field of the Invention

The invention relates generally to tools for preparing a hole to receive an implant or fixture, and more particularly to rotary osteotomes and methods implemented thereby for expanding an osteotomy or hole in other materials to receive an implant or other anchoring device.

### Description of Related Art

An implant is a medical device manufactured to replace a missing biological structure, to support a damaged biological structure, or to enhance an existing biological structure. Bone implants are implants of the type placed into the bone of a patient. Bone implants may be found throughout the human skeletal system, including but not limited to dental implants in the jaw to replace a lost or damaged tooth, joint implants to replace a damaged joint such as in hips and knees, and reinforcement implants installed to repair fractures and remediate other deficiencies like pedicle screws used in spinal stabilization, and so forth. The placement of an implant often requires a hole preparation, taking care to prevent burning or pressure necrosis of the bone. After time to allow integration of the implant into the bone or other host material, sufficient healing will enable rehabilitation therapy or return to normal use or perhaps the placement of a restoration or brace or other attachment feature.

There are several known ways to form a receiving hole for an implant. Since the early days of implantology, for example, osteotomies have been prepared using standard drills that look and handle much like drills designed for use in industrial boring applications. These drill designs have proven to be moderately functional for dental and medical applications, however noticeably imperfect. Implant success rates have been satisfactory over time, but osteotomy preparation techniques have still been lacking for various reasons. Standard boring drill designs used in prior art dental and medical implantology are designed to excavate a hole in the bone to create space for the implant to be placed, just like a boring drill designed for carpentry. Standard boring drill designs, in twist or fluted shapes, cut bone effectively but typically do not produce a clean, precise circumferential osteotomy. Osteotomies may become elongated and elliptical due to chatter because the boring drills are very aggressive cutters. In circumstances where the osteotomy is imperfectly round, the implant insertion torque may be reduced, leading to poor primary stability and potential lack of integration. Osteotomies drilled into narrow bone locations may produce dehiscence, buccally or lingually, which also reduces primary stability and might require an additional bone grafting procedure, thereby adding cost and healing time to the treatment.

In recent times, a novel biomechanical bone preparation technique called “osseodensification” has gained widespread acceptance in the global dental community. The osseodensification technique, which was pioneered by the Applicant of this invention, is a high-speed rotary technique that preserves the host bone throughout the hole-forming procedure. Bone retention has been proven to increase implant stability and accelerate regenerative bone growth. In some instances, osseodensification is now considered a preferred standard of care. Examples of osseodensification can be seen in U.S. Pat. No. 9,028,253, issued May 12, 2015, and in U.S. Pat. No. 9,326,778, issued May 3, 2016, and PCT Publication No. WO 2020/210442, published Oct. 15, 2020. The entire disclosures of these references are hereby incorporated by reference and relied upon to the extent permitted by each national jurisdiction.

Generally described, osseodensification is a procedure for enlarging an osteotomy using a specially-designed, multi-fluted, rotary osteotome. An example of a suitable rotary osteotome is described in the above-mentioned U.S. Pat. No. 9,326,778. Rotary osteotomes for dental applications are marketed as Densah® Burs through Versah, LLC of Jackson, Michigan USA, a licensee of the Applicant.

Unlike traditional boring techniques, osseodensification does not excavate bone tissue. Rather, bone tissue is simultaneously compacted and auto-grafted in an outwardly expanding direction, somewhat akin to a traditional hammered osteotome but without the trauma and other limitations of that percussive technique. When rotated at high speed in a reversed, non-cutting direction with steady external irrigation, these rotary osteotomes form a strong and dense layer of bone tissue along the walls and base of the osteotomy. Dense compacted bone tissue produces stronger purchase for the implant and may facilitate faster healing.

Briefly, an example of dental implantology may be used to illustrate the general principles of the osseodensification technique. The osteotomy site is first prepared with a precursor pilot hole drilled with a small, e.g., 1.5 mm, standard medical-grade twist drill, tri-lobed drill or other boring tool. (Of course, the circumstances of any given surgical application, whether dental or non-dental in nature, will dictate the size of precursor pilot hole and other characteristics of the operation.) The precursor pilot hole is drilled to a predetermined depth. Using a rotary osteotome designed for osseodensification, the surgeon decides whether to enlarge the precursor pilot hole either by densifying or cutting, taking into account situational factors which may include hardness of the bone, final intended osteotomy/implant size, local width of bone formation, and other relevant factors.

If the surgeon decides to enlarge the precursor pilot hole by cutting, the specially designed rotary osteotome is rotated in a cutting direction at high speed. High speed is defined as generally above 200 RPM for rotary osteotomes in the range of about 1.5 mm to 6 mm in diameter. The rotary osteotome is advanced into the precursor pilot hole, often with a gentle pumping motion and abundant irrigation. On its descent, the working edges of the rotary osteotome cut bone materials into small chips or particles, which accumulate in the flutes. The bone particles are subsequently discarded or collected/harvested if desired for later use. The osteotomy can likewise be further enlarged by cutting (or densifying) in one or more subsequent operations using progressively larger rotary osteotomes.

If instead of cutting the surgeon prefers to enlarge the precursor pilot hole by densifying, the same rotary osteotome is used but instead rotated in a non-cutting direction at high speed. If the rotary osteotome is designed so that its cutting direction is clockwise (as is typical with most twist drills), then the non-cutting direction for that same rotary osteotome would be counter-clockwise. I.e., the non-cutting or densifying direction is the reverse of the cutting direction. When densifying, the surgeon advances the counter-spinning rotary osteotome into the precursor pilot hole (or a precursor hole formed by a previous expansion operation like that described in the preceding paragraph), together with copious irrigation. Downward pressure applied by the surgeon is needed to keep the working edges of the rotary osteotome in contact with the bone surface inside the osteotomy, often with the above-mentioned gentle bouncing motion to modulate the pressure and thereby avoid over-heating and over-straining of bone tissue. The harder the surgeon pushes the rotary osteotome into the osteotomy, the more pressure is exerted laterally, both mechanically and through hydrodynamic effects enabled by the concurrent irrigation. Care is taken to maintain alignment between the longitudinal axis of the rotary osteotome and the bore axis of the osteotomy at all times. Once the rotary osteotome has reached the intended depth, enlargement with that rotary osteotome is complete. The osteotomy can then be further enlarged by densifying with one or more subsequent operations using progressively larger rotary osteotomes following similar procedures.

Biomechanical, histological, as well as clinical histological validation studies of the osseodensification technology have concluded that osseodensification facilitates bone expansion, increases implant stability and creates a densification layer around the preparation site by compacting and autografting bone particles along the entire depth of the osteotomy.

Although described up to now in the context of medical applications, these same techniques are applicable to non-bone materials. Some industrial applications, including those which require the placement of screwed anchors into other organic materials, such as wood, or into and other non-organic materials such as foamed and cellular compositions of polymers and/or metals, may be accepting of and benefit from the general principles of this technology.

Osseodensification is a relatively new field. As with any emerging technology, new and improved tools and techniques are expected as the technology matures. Furthermore, there is a continuing need to improve the efficiency and effectiveness of surgical operations. Therefore, any improvements in osseodensification tools and/or techniques that advance the base technology, and that improve efficiency and efficacy, will be desired.

### BRIEF SUMMARY OF THE INVENTION

According to a first aspect of the present invention, a rotary osteotome is adapted for preparing an osteotomy with osseodensification effects. The rotary osteotome comprises a shank that establishes a longitudinal axis of rotation. The shank has an upper end and a lower end. A body extends axially from the lower end of the shank. The body has an apical end that is remote from the shank. A plurality of flutes are disposed about the body. Each flute has a working edge. The apical end has at least two lips that are arranged circumferentially about the longitudinal axis in at least one adjacent pair. The lips form primary cut/grind edges. A debris control surface extends between the adjacent pair of lips. A valley passes though the longitudinal axis and between the adjacent pair of lips. The valley intersects at least a portion of the debris control surface and establishes a plurality of secondary cut/grind edges at the intersections with the debris control surface.

According to a second aspect of the present invention, a rotary osteotome is adapted for preparing an osteotomy with osseodensification effects. The rotary osteotome comprises a shank that establishes a longitudinal axis of rotation. The shank has an upper end and a lower end. A body extends axially from the lower end of the shank. The body has an apical end that is remote from the shank. A plurality of flutes are disposed about the body. Each flute has a working edge. The apical end has at least two lips arranged circumferentially about the longitudinal axis in at least one adjacent pair. The lips form primary cut/grind edges. A debris control surface extends between the adjacent pair of lips. The debris control surface comprises at least one trailing flank that extends from one lip in the adjacent pair. At least one valley passes though the longitudinal axis while bisecting the adjacent pair of lips. The valley has upswept sidewalls that truncate a central portion of the lips in the adjacent pair. The valley establishes a plurality of secondary cut/grind edges at the interface of the trailing flank.

According to a third aspect of the present invention, a method is provided for enlarging a precursor hole to receive a screw-in fixture. The method comprises the steps of rotating a rotary osteotome at greater than 200 RPM, positioning the rotating rotary osteotome over a smaller precursor hole. The precursor hole has an interior surface that extends between a generally circular entrance and a closed bottom. The interior surface of the precursor hole has a conically tapered sidewall. Enlarging the precursor hole by forcibly pushing the rotating body to the bottom of the precursor hole while concurrently autografting produced bone debris particles into the side walls of the enlarged hole. The enlarging step includes bathing the rotary osteotome with irrigating fluid. Forming a button of bone debris at the bottom of the enlarged hole. And the method concludes by withdrawing the rotary osteotome from the enlarged hole while the button of bone debris remains at the bottom of the enlarged hole.

The novel valley feature of this invention is a relief in the tip that yields a button-like amount of bone chips or fragments at the center of the osteotomy bottom. These left-behind bone fragments provide bone growth accelerant to facilitate new bone ingrowth and osseointegration of a subsequently installed anchor. Furthermore, the valley has a beneficial effect on the flow of bone particles from the surfaces the apical end into the flutes. In particular, the valley enables debris to be more equally distributed among the flutes, resulting in smoother operation of the osteotome with less rotary imbalance. The valley forms secondary cut/grind edges at its intersections with the debris control surface that generate bone debris more rapidly than previous designs which replied entirely upon the primary cut/grind edges established by the lips. The bone debris is efficiently carried away from the apical end, thereby reducing the possibility of heat-induced and/or pressure-induced damage to the bone particles. The novel features of the invention produce bone debris with less direct surface contact with apical bone, with less vertical pressure effort for the surgeon to advance such tool and with less trauma for the patient. The present invention better protects the vitality and health of the bone debris thereby facilitating autografting efficacy.

### BRIEF DESCRIPTION OF THE SEVERAL VIEWS OF THE DRAWINGS

These and other features and advantages of the present invention will become more readily appreciated when considered in connection with the following detailed description and appended drawings, wherein:

FIG. 1 depicts an exemplary application of the present invention at an edentulous (without teeth) jaw site in the process of forming an osteotomy to receive an implant, the process involving use of a rotary osteotome according to an embodiment of the invention;

FIG. 2 is a perspective view of a rotary osteotome according to an embodiment of the invention;

FIG. 3 is a front elevation view of the rotary osteotome of FIG. 2;

FIG. 4 is a top view of the rotary osteotome of FIG. 2;

FIG. 5 is an apical end view of the rotary osteotome taken generally along lines 5-5 of FIG. 4;

FIG. 6 is an enlarged view of the apical end of the rotary osteotome taken from a first perspective;

FIG. 7 is an enlarged view of the apical end of the rotary osteotome taken from a second perspective;

FIG. 8 is an enlarged view of the apical end of the rotary osteotome taken from a third perspective;

FIG. 9 is an enlarged view of the apical end of the rotary osteotome taken from a fourth perspective;

FIG. 10A is a simplified view showing the apical end of the rotary osteotome descending toward the base of an osteotomy;

FIG. 10B is a further progression of FIG. 10A showing the apical end of the rotary osteotome in contact with the base of the osteotomy;

FIG. 11A is an enlarged view of the area circumscribed at 11A in FIGS. 10B, and including the build-up and upward migration of drilling debris;

FIG. 11B is a further progression of FIG. 11A showing the apical end of the rotary osteotome lifted away from the base of the osteotomy, leaving a small button of drilling debris at the base of the osteotomy;

FIG. 12 is an apical end view of an alternative embodiment showing the outward migration of drilling debris into the several flutes;

FIG. 13 is a fragmentary front elevation view of a first alternative embodiment of the rotary osteotome;

FIG. 14 is an end view as taken along lines 14-14 of FIG. 13;

FIG. 15 is a fragmentary front elevation view of a second alternative embodiment of the rotary osteotome; and

FIG. 16 is an end view as taken along lines 16-16 of FIG. 15.

### DETAILED DESCRIPTION OF THE INVENTION

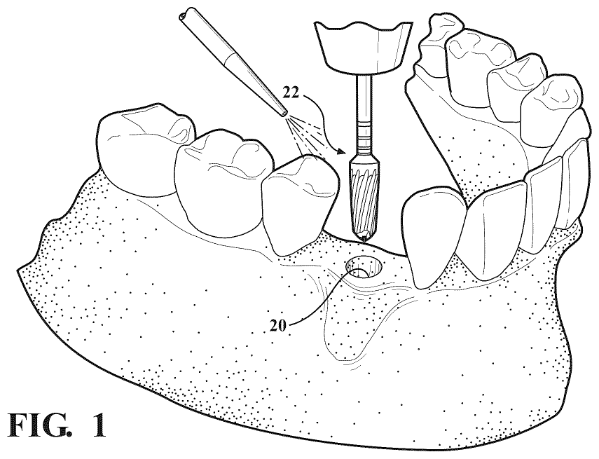

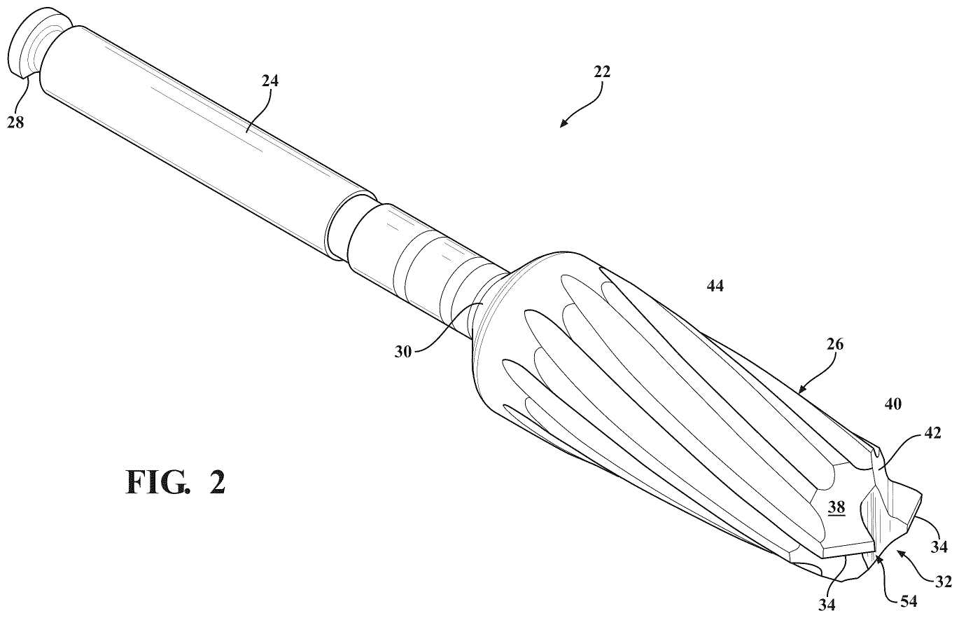

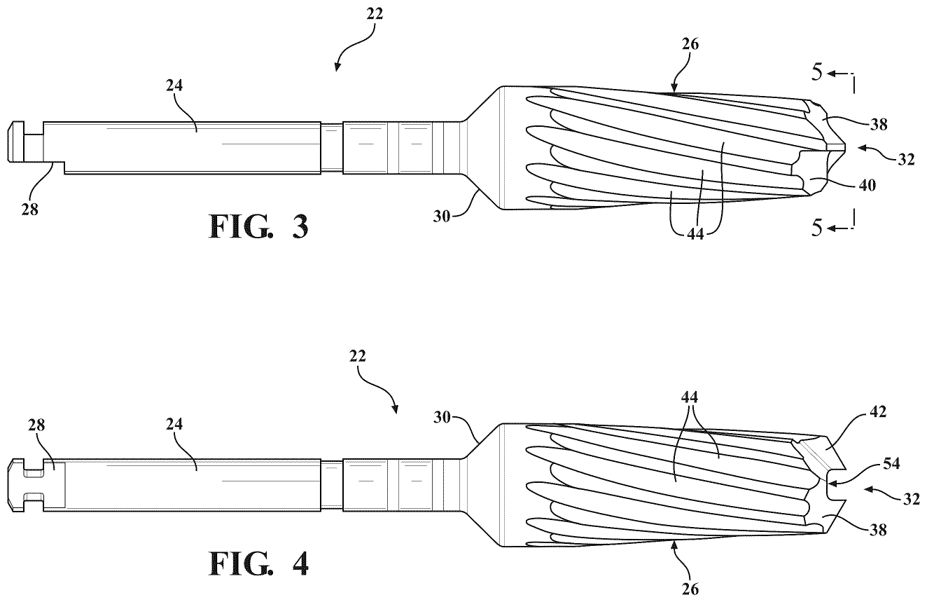

Referring to the figures, wherein like numerals indicate like or corresponding parts throughout the several views, FIG. 1 shows the example of a dental procedure to receive an implant (not shown). It will be understood that this invention is not limited to dental applications, but instead may be applied across a wide spectrum of orthopedic applications. Human applications are typical, but animal applications are equally plausible and not outside the scope of this invention. Furthermore, the invention is not limited to bone applications but may be used to prepare holes in living organic, non-living organic and non-organic materials for a wide variety of applications. In FIG. 1, an edentulous (without teeth) jaw site has been prepared after the manner previously described to achieve a fully formed osteotomy 20 (FIG. 2) ready to receive a screw-in implant (not shown) or other anchoring device. In this example, the osteotomy 20 is formed using an improved rotary osteotome 22 according to the present invention. The rotary osteotome 22 is also known in the art as a bur, a burr and a drill.

The surgeon locks or otherwise installs the rotary osteotome 22 into the drill motor (FIG. 1) and sets the rotational direction to a non-cutting direction, which in the illustrated example is counter-clockwise as viewed looking along the length of the osteotome 22 from its connection with the drill motor toward its leading tip. The leading tip is also known as the apical end. Those of skill in the art will understand how to reconfigure the osteotome 22 so that its non-cutting rotational direction is instead clockwise. Although the surgeon may vary the rotational speed of the osteotome 22 according to the needs of the situation, experimental results indicate that high rotation speeds, i.e., greater than about 200 RPM, and torque settings between about 5-80 Ncm will provide satisfactory results. High speed rotation is considered anything above about 200 RPM for rotary osteotome 22 diameters in the range of about 1-7 mm. Upper ranges for these relatively small diameter rotary osteotomes may reach about 2000 RPM. More preferably, rotation speeds between about 600-1800 RPM and torque settings between about 20-80 Ncm have been found to provide satisfactory results. And still more preferably, rotation speeds in the range of 800-1500 RPM and torque settings of about 50 Ncm provide satisfactory results. Speeds suggested here apply in context to the exemplary dental applications. Relatively large diameter rotary osteotomes 22 used for large-bone orthopedic applications, such as femurs, can be larger than 7 mm and may require slower rotational speeds than rotary osteotomes 22 used for smaller bone applications due to tangential velocity considerations at the working edges. That is to say, as a guiding principle for large diameter rotary osteotomes 22, it may be advantageous to maintain tangential velocity as measured at the outer edges between about 0.02-0.6 m/s to create a suitable compression wave in the bone needed to accomplish osseodensification. A continuous flow of irrigating fluid is used throughout the procedures.

Turning now to FIG. 2, an osteotome 22 according to an embodiment of this invention is shown including a shank 24 and a body 26. The shank 24 has an elongated cylindrical shaft that establishes a longitudinal axis of rotation for the rotary osteotome 22. A drill motor engaging interface 28 is formed at the distal upper end of the shaft for connection to a standard drill motor. The particular configuration of the interface 28 may vary depending on the type of drill motor used, and in some cases may even be merely a smooth portion of the shaft against which the jaws of a collet grip. The body 26 joins to the lower end of the shank 24, which joint may be formed with a tapered or domed transition 30. The transition 30 acts something like an umbrella as the surgeon irrigates with water during a procedure. The gentle transition 30 facilitates the flow of irrigating fluid onto the osteotomy site while the osteotome 22 is spinning.

The body 26 preferably has a conically tapered profile decreasing from a maximum diameter adjacent the shank 24 and transition 30 to a minimum diameter adjacent an apical end 32. However, in some contemplated embodiments the body may be non-tapered (i.e., cylindrical). The apical end 32 is thus remote from the shank 24. The working length or effective length of the body 26 is related to the application for which it is intended, as well as the taper angle of the body 26. Taper angles between about 1° and 10° (or more) are possible depending upon the application. In many cases, the taper angle will be between about 1.5°-5°. More preferably taper angles between about 2°-3° will provide satisfactory results for dental applications when the body 26 length is between about 11 mm and 15 mm.

The apical end 32 is defined by at least two, but very possibly more, lips 34. The lips 34 are in fact edges that are arranged circumferentially about the longitudinal axis in adjacent pairs. The lips 34 form the primary cut/grind edges at the apical end 32 of the osteotome 22. In the illustrated examples, cutting occurs when the osteotome 22 is rotated in a cutting direction and grinding when it is rotated in a non-cutting, or condensing, direction. However, it is possible to configure the apical end 32 so that the lips 34 will be arranged to grind in the cutting direction and cut in condensing direction because the cutting vs. non-cutting direction is defined by the flutes/working edges (described below) and not by the configuration of the apical end 32.

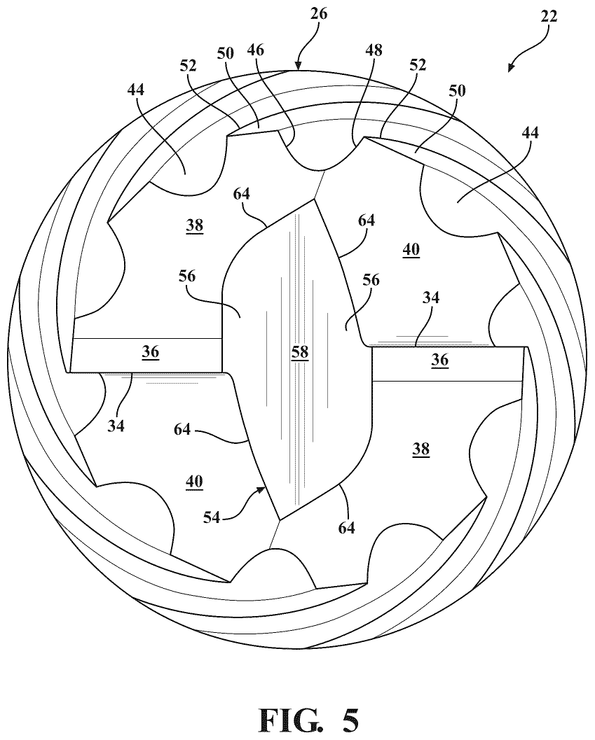

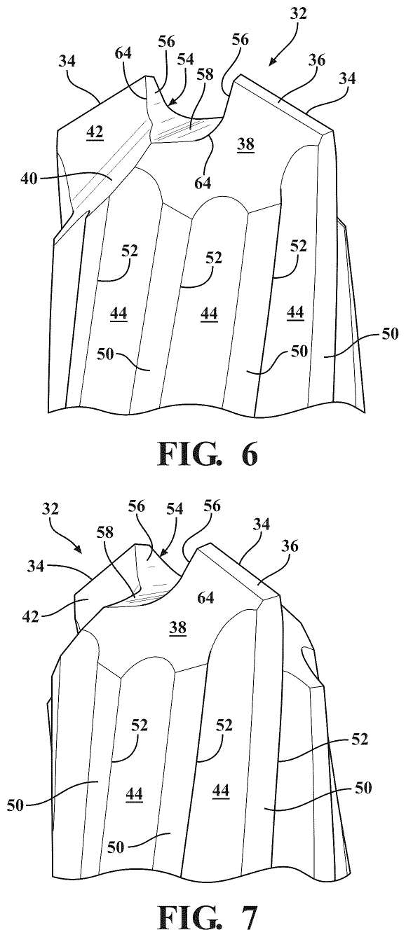

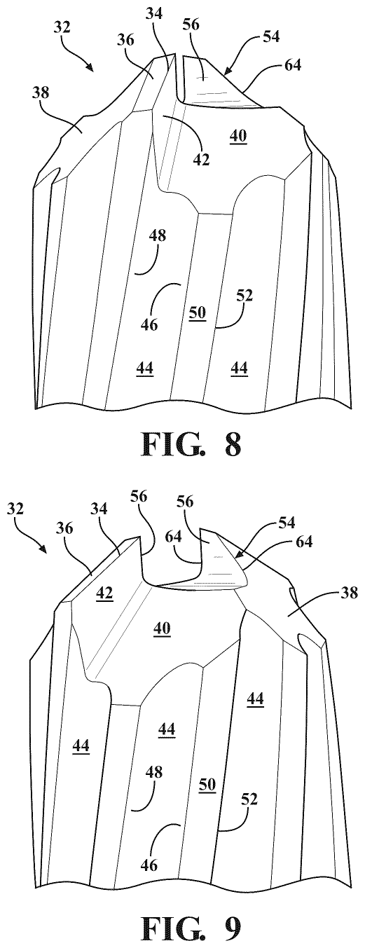

The illustrated examples show an osteotome 22 having two lips 34, which constitute a single pair of lips 34. In this case, the lips 34 are disposed on opposite sides of the apical end 32, and may or may not lie within a common plane. As shown in the end view of FIG. 5, the lips 34 may be slightly offset (in terms of a direct diametrical alignment). The lips 34 angle upwardly and outwardly (radially) from the apical end 32 at a point angle. The point angle of the lips 34 may be varied to optimize performance for the application. The term “point angle” is taken from that known in common industrial machining fields to be the included angle between the lips 34. Point angles may range between about 60° (very pointed) and 180° (very blunt). That is to say, the at least two lips are angled relative to one another in an arc that passes through the longitudinal axis, with the measure of this arc being the point angle. Or in another example, an imaginary right circular cone aligned with the longitudinal axis and having an interior angle measure equal to the point angle will concurrently touch the full lengths of all lips, allowing of course for normal manufacturing tolerances. In the illustrated examples, the lip angle is approximately 120° measured between the two opposing lips 34.

In embodiments where the osteotome has more than two lips 34, it is more likely that all lips 34 will share the same point angle. Using the imaginary cone example just mentioned, all of the lips will concurrently lay along the sides of the cone, allowing of course for normal manufacturing tolerances. It is contemplated however that for embodiments where the osteotome has four lips, the two opposing lips could if desired have a different point angle than the other two opposing lips so as to achieve certain desire cutting or grinding attributes. For reasons that will become apparent, osteotomes 22 having an odd number of lips (e.g., 3, 5, etc.) are possible but not preferred over those having an even number of lips (e.g., 2, 4, 6, etc.). And so, an osteotome 22 configured with four lips 34 would have four adjacent pair of lips 34 arranged circumferentially about the longitudinal axis-one pair for each quadrant. An osteotome 22 configured with six lips 34 would have six adjacent pair of lips 34, and so forth.

Aside from having at least two lips 34, the apical end 32 is designed with a debris control surface between adjacent pairs of lips 34 that will facilitate movement and/or congregation of bone debris in an intentional way. In the two-lip embodiment show in the accompanying illustrations, the adjacent pair of lips 34 are approximately 180-degrees apart. Such an embodiment could be configured therefore with two debris control surfaces, each extending the entire, or at least a majority of, distance between the lips 34 on both sides, thus each spanning an arc of about or nearly 180-degrees.

In certain contemplated embodiments where the osteotome is configured with more than two lips, a debris control surface will extend between at least one pair of adjacent pair of lips, or more preferably between each adjacent pair of lips. In for example an embodiment where the osteotome 22 is configured with four lips, it would be expected that the lips are set approximately 90-degrees apart from one another. There would be at least one, but ordinarily four, debris control surfaces with each debris control surface spanning an arc of about or nearly 90-degrees between the adjacent pairs of lips 34.

The debris control surfaces can be fashioned in a wide variety of ways. In some contemplated embodiments, the debris control surface could be a flat or curvaceous ramp-like feature extending between the one or more lips 34. In the examples provided however, the debris control surface comprises a multi-faceted formation, which includes a first trailing flank 36 that is pitched from a respective one of the lips 34 at a first angle. The first trailing flank 36 is shown as being generally planar, however non-planar shapes are certainly possible. To be clear, the presence or absence of a discrete first trailing flank 36 behind the lip 36 is a matter of design choice. The first angle may be varied to optimize performance for the application. In practice, the first angle may be between about 30° and 60°, the measurement of which is accomplished by rotating a 2D image so that the first trailing flank 36 appears as a line (i.e., is perfectly perpendicular to the plane of the paper or screen on which it appears). At this orientation, the first angle is measured directly from the longitudinal axis of the osteotome 22.

In most cases, the first trailing flanks 36 are set relative to their respective lips 34 so that when the osteotome 22 is rotated in use, the first trailing flanks 36 either lead or follow their respective lips 34. As mentioned previously, the apical end 32 can be formed in various ways, and the cutting/non-cutting directions of the osteotome 22 are defined by the flutes and working edges (described below). In examples illustrated in the accompanying drawings, the first trailing flanks 36 lead their respective lips 34 when the osteotome 22 is said to be turning in a non-cutting direction for the densifying mode; and conversely when the first trailing flanks 36 follow their respective lips 34, the osteotome is said to be turning in a cutting direction where the lips 34 cut or slice bone on descent. In the densifying direction, the first trailing flanks 36 form, in effect, a large negative rake angle for the lips 34 to minimize chip formation and shear deformation in the bone (or other host material) at the point of contact with the lips 34.

However, the foregoing statements are generalizations and not necessarily the case in all embodiments. It is contemplated that in some applications, it may be desirable to reconfigure the apical end so that the first trailing flanks 36 trail their respective lips 34 when the osteotome 22 is turning in a non-cutting direction for the densifying mode. Indeed, many variations are possible and within the contemplated scope of this invention.

To say again, the debris control surface of the apical end 32 can be designed in a wide variety of ways. In the examples provided, the debris control surface includes a second trailing flank 38 that is formed adjacent to, and falls away from, each first trailing flank 36 at a second angle. The second trailing flank 38 is shown as being generally planar, however non-planar shapes are possible. To be clear, the presence or absence of a second trailing flank 38 behind the first trailing flank 36 is a matter of design choice. The second angle would be smaller than the first angle and optimized for the application. In practice, the second angle is typically less than about 55°. In an example where the first trailing flanks 36 are formed at 45° (relative to the axis), the second trailing flanks 38 may be 40° or less. Measurement of the second angle would be accomplished in a manner similar to that described for the first angle.

The debris control surface of the apical end 32 may also be formed with relief pockets 40 adjacent to each second trailing flank 38. To be clear, the presence or absence of a relief pocket 40 is a matter of design choice; the osteotome 22 can be made to function with satisfaction without a relief pocket 40. Or said another way, the relief pockets 40 could in some embodiments be integrated with the second trailing flanks 38 such that it is not convenient to distinguish one from the other. In particular, embodiments where the osteotome is configured with more than two lips may not be able to include a relief pocket 40 due to space constraints. Each relief pocket 40 pitches away from its associated second trailing flank 38 at a third angle. The third angle is smaller than the second angle, as determined by a measurement technique similar to that described for the first angle. In an example where the second trailing flanks 38 are formed at 40° (relative to the axis), the relief pockets 40 (i.e., the third angle) may be 30° or less. Each relief pocket 40 is disposed in a sector of the apical end 32 between a second trailing flank 38 and a lip 34.

Another element of the debris control surface is a lip face 42 that extends between the relief pocket 40 and the adjacent lip 34. The lip face 42 can be generally axially disposed, or of some other configuration. When the osteotome 22 is rotated in the cutting direction, a significant amount of bone chips collect in the relief pocket 40 regions. When the osteotome 22 is rotated in the densifying direction, little to no bone chips collect in the relief pocket 40 regions.

A plurality of grooves or flutes 44 are disposed about the body 26. The flutes 44 may or may not have common axial length and radial depths. I.e., it is possible that the flutes 44 could, in some configurations, not all be identical. The flutes 44 are preferably, but not necessarily, equally circumferentially arranged about the body 26. The diameter of the body 26 may influence the number of flutes 44. As an example, bodies 42 in the range of about 1-3 mm may be formed with three or four flutes; bodies 42 in the range of about 3-4 mm may be formed with five or six flutes; bodies 42 in the range of about 4-5 mm may be formed with seven or eight flutes; and bodies 42 in the range of about 5-6 mm may be formed with nine or ten flutes. And even larger bur diameters, e.g., 6-7 mm could, if desired, have even more flutes. Of course, number of flutes 44 may be varied more or less from the examples given here in order to optimize performance and/or to better suit the desired application.

In the illustrated embodiment, the flutes 44 are formed with a helical twist. If the cutting direction is in the right-hand (clockwise) direction, then preferably the helical spiral is also in the right-hand direction. This RHS-RHC configuration is shown throughout the Figures, although it should be appreciated that a reversal of cutting direction and helical spiral direction (i.e., to LHS-LHC) could be made if desired with substantially equal results. It is also possible to configure the osteotome with a LHS-RHC or RHS-LHC configuration. That is, it may be desirable to arrange the helical spiral so that the osteotome 22 is drawn into the hole rather than pushed out of the hole in a densifying mode of operation.

Each flute 44 has a densifying face 46 and an opposing cutting face 48. A rib-like land 50 is formed between adjacent flutes 44, in alternating fashion. Thus, a four-fluted osteotome 22 will have four lands 50, a ten-fluted osteotome 22 will have ten interleaved lands 50, and so forth. Each land 50 extends (circumferentially) between the densifying face 46 of the flute 44 on one side and the cutting face 48 of the flute 44 on its other side. The sharp interface between each land 50 and its associated cutting face 48 is referred to as a working edge 52. Depending on the rotational direction of the osteotome 22, the working edge 52 either functions to cut bone or compact bone. That is, when the osteotome is rotated in the cutting direction, the working edges 52 slice and excavate bone (or other host material). Typically, in the cutting direction, the working edges 52 will have a light tendency to pull the osteotome 22 deeper into the osteotomy 20. When the osteotome is rotated in the densifying (non-cutting) direction, the working edges 52 compress and radially displace bone (or other host material) with little to no cutting. This compaction and radial displacement is exhibited as gentle pushing of the osseous structure laterally outwardly in a condensation mechanism. Typically, in the non-cutting direction, the working edges 52 will have a light tendency to resist penetration of the osteotome 22 into the osteotomy 20.

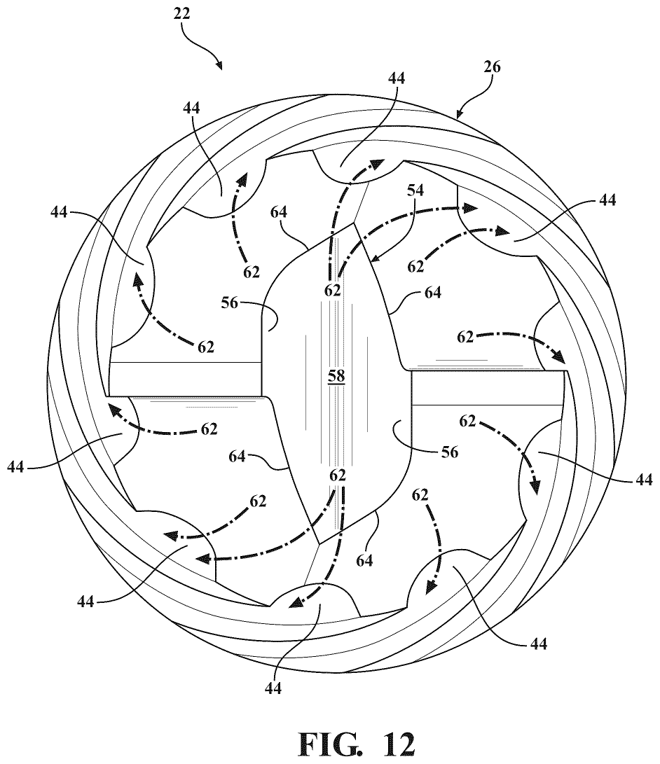

The working edges 52 shown throughout most of the illustrations are substantially margin-less, in that the entire portion of each land 50 is cut away behind the working edge 52 to provide complete clearance. Margin-less working edges 52 are preferable, however in some cases the lands 50 could be designed to operate as margins. The alternative embodiment of FIG. 12 shows lands 50 that also serve as margins for the working edges 52. In standard prior art twist drills, margins are commonly incorporated behind the working edge to help guide the drill in the hole and maintain the drill diameter. In a margin-less design, the primary taper clearance angles, i.e., the angle between a tangent of the working edge 52 and each land 50, may fall anywhere between about 1° and 35° (or larger) depending upon the application and, possibly, on the diameter of the body 26. Primary taper clearances between about 5° and 20° have been found effective for the body 26 diameters between about 1-7 mm. Of course, the primary taper clearance angles may be varied more or less than the examples given here to optimize performance and/or to better suit the application. For cases where the lands 50 form margins behind the working edges 52 as in FIG. 12, the primary taper clearance angle would be effectively 0°.

As mentioned above in connection with the angle of the helical twist, for the standard RHS-RHC and LHS-LHC configurations, the working edges 52 can be seen turning away from the densifying direction as the conically tapered profile of the body 26 decreases in diameter. In other words, when the densifying direction is counter-clockwise, the helical twist of the working edges 52 winds in the counter-clockwise direction when viewed from the top of the body 26 looking toward its apical end 32. Or conversely, when viewed from the apical end 32 looking toward top of the body 26, the twist will appear to be in the clockwise direction. Thus, when the densifying direction is counter-clockwise, the working edges 52 will turn away from the densifying direction when all of the land 50 and flutes 44 orbit counter-clockwise about the longitudinal axis as one traces each land 50 and flute 44 downwardly toward the apical end 32.

The cutting face 48 establishes a rake angle for each respective working edge 52 when rotated in the cutting direction. A rake is an angle of slope measured from the leading face of the working edge 52 to an imaginary line extending perpendicular to the surface of the worked object (e.g., inner bone surface of the osteotomy). Rake angle is a parameter used in various cutting and machining processes, describing the angle of the cutting face relative to the work. Rake angles can be: positive, negative or zero. Rake angles from about zero degrees (0°) and above will establish a crisp cutting edge 52 well-suited to cut/slice bone when the osteotome 22 is rotated in the cutting direction. However, it has been discovered that the cutting functionality of the rotary osteotome 22 can be improved by changing the rake angle of the cutting face 48 below zero degrees (0°). In particular, cutting face 48 rake angles between about 0° and about −65° (i.e., negative rake) can be useful in certain documented applications.

When the osteotome 22 is rotated in the densifying mode, a heel-side angle is established between the working edge 52 and the land 50, which as previously stated may lie at a large negative rake angle in the order of about 55°-90°, which is the compliment of the primary taper clearance angle. From a definitional point of view, the heel-side angle is a rake angle of the working edge 52 when rotated in a densifying direction. However, to avoid confusion with respect to the rake angle of the cutting face 48, the heel-side angle terminology is adopted. The large negative (or zero degree) heel-side angle applies outward pressure at the point of contact between the wall of the osteotomy 20 and the working edge 52 to create a compression wave ahead of the point of contact, loosely akin to spreading butter on toast. Osscodensification may also be loosely compared to the well-known process of burnishing metal to improve metal surface quality.

Downward pressure applied by the surgeon is needed to keep the working edge 52 in contact with the bone surface of the osteotomy 20 as it is being expanded. That is, pressure is needed to generate and propagate a compression wave in the bone that begins when the contact stresses exceed the yield strength of the host material. This is aided by the complimentary taper effect of the osteotomy 20 and body 26 to create lateral pressure (i.e., in the outward direction of expansion). The harder the surgeon pushes the rotary osteotome 22 into the osteotomy 20, the more pressure is exerted laterally. This gives the surgeon complete control of the expansion rate irrespective to a large degree on the rotation speed of the osteotome 22, which is a factor underlying the short learning curve required to master the osseodensification technique. Thus, the intensity of the compaction effect depends chiefly on the amount of force exerted on the osteotome 22, which is controlled by the surgeon. The greater the force exerted, the quicker expansion will occur.

As each working edge 52 drags across the bone, the applied forces can be decomposed into two components: one normal to the bone's surface, pressing it outwardly, and the other tangential, dragging it along the inner surface of the osteotomy 20. As the tangential component is increased, the working edge 52 will start to slide along the bone. At the same time, the normal force will deform the softer bone material. If the normal force is low, the working edges 52 will rub against the bone but not permanently alter its surface. The rubbing action will create friction and heat, but this can be controlled by the surgeon by altering, on-the-fly, the rotation speed and/or pressure and/or irrigation flow. Because the body 26 of the osteotome 22 is tapered, the surgeon may at any instant during the surgical procedure lift the working edges 52 away from contact with the surface of the bone to allow cooling. This can be done in a controlled “bouncing” fashion where pressure is applied in short bursts with the surgeon continuously monitoring progress and making fine corrections and adjustments. As the surgeon-applied downward force increases, eventually the stresses in the bone's surface exceed its yield strength. When this happens, the working edges 52 will plow through the surface and create a trough behind. The plowing action of the working edges 52 thus progressively enlarges the osteotomy until the rotary osteotome 22 reaches the desired depth, at which time a different larger rotary osteotome 22 must be used to achieve further expansion if desired.

The elastic properties of bone are well-documented. If the load imposed by the surgeon does not exceed the bone's ability to deform elastically, the bone will promptly return to its initial (un-deformed) condition once the stress is removed. On the other hand, if the load imposed by the surgeon exceeds the bone's ability to deform elastically, the bone will deform and change shape permanently by plastic deformation. In bone, the permanent change in shape may be associated with micro-cracks that allow energy release, a compromise that is a natural defense against complete fracture. If these micro-cracks are small, the bone remains in one piece while the osteotomy expands. When bone is subjected to stress in the region between its yield point and its ultimate tensile strength, the material experiences strain hardening. Strain hardening, also known as work hardening or cold working, is the strengthening of a ductile material by plastic deformation. This strengthening occurs because of dislocation movements and dislocation generation within the crystal structure of the material—which for bone corresponds with the dislocation of the cross-links between collagen fibers in the bone tissue. The material tends to experience necking when subjected to stress in the region between its ultimate tensile strength and the point of fracture.

The direction of helical twist can be designed to play a role in contributing to the surgeon's control so that an optimum level of stress (in the strain hardening zone) can be applied to the bone throughout the expansion procedure. In particular, the RHS-RHC configuration described above, which represents a right-hand spiral for a right-hand cutting direction (or alternatively an LHS-LHC configuration, not shown) applies a stress that provokes a beneficial opposing axial reaction force in the host bone when the osteotome 22 is continuously rotated at high speed in a densifying direction and concurrently forcibly advanced (manually by the surgeon) into an osteotomy 20. In other words, if the surgeon operating the osteotome 22 is pushing the osteotome 22 downwardly into an osteotomy 20, then the opposing axial reaction force works in the opposite direction to push the osteotome upwardly. The opposing axial reaction force is the vertical (or perhaps more accurately the “axial” vis-à-vis the longitudinal axis) component of the reaction force (R) that is applied by the bone against the full length of the working edges 52 of the osteotome 22. An opposing axial reaction force is also created by the effectively large negative rake angle at the lips 34 when the osteotome 22 is rotated in a densifying direction. Those of skill in the art will appreciate alternative embodiments in which the opposing axial reaction force is created by either the configuration of the lips 34 alone or of the working edges 52 alone rather than by both acting in concert as in the preferred embodiment.

For a surgeon to advance the apical end 32 toward the bottom of the osteotomy 20 when the osteotome 22 is spinning in the densifying direction, he or she must push against and overcome the opposing axial reaction forces in addition to supplying the force needed to plastically displace/expand the bone as described above. The osteotome 22 is designed so that the surgeon must continually work, as it were, against the opposing axial reaction forces to expand the osteotomy 20 by compaction, i.e., when in the densifying mode. Rather than being a detriment, the opposing axial reaction forces are a benefit to the surgeon by giving them greater control over the expansion process. Because of the opposing axial reaction forces, the osteotome 22 will not be pulled deeper into the osteotomy 20 as might occur with a standard “up cutting” twist drill or burr that is designed to generate a tractive force that tends to advance the osteotome toward the interior of the osseous site. So-called “up-cutting” burrs have the potential to grab and pull the burr more deeply into the osteotomy, which could lead to inadvertent over-penetration.

In the densifying mode, the intensity of the opposing axial reaction forces is always proportional to the intensity of force applied by the surgeon in advancing the body 26 into the osteotomy 20. This opposing force thus creates real-time haptic feedback that is intuitive and natural to inform the surgeon whether more or less applied force is needed at any given instant. This tactile feedback takes full advantage of the surgeon's delicate sense of touch by applying reaction forces directly through the osteotome 22. In this densifying mode, the mechanical stimulation of the opposing axial reaction forces assists the surgeon to better control the expansion procedure on the basis of how the bone (or other host material) is reacting to the expansion procedure in real time.

Thus, the controlled “bouncing” or “pumping” action described above is made more effective and substantially more controllable by the opposing axial reaction forces so that the surgeon can instinctively monitor progress and make fine corrections and applied pressure adjustments on-the-fly without losing control over the rate of expansion. The tactile feedback from the opposing axial reaction forces allows a surgeon to intuitively exert stress on the bone material so that its strain response preferably resides in the strain hardening zone. In any event, the surgeon will endeavor to maintain the stress (as generated by the force he or she applies through the rotating osteotome 22 above the elastic limit and below the point of fracture. Of course, until the applied stress passes its elastic limit, the bone will not permanently deform at all; and to apply stress beyond the point of fracture will cause the bone (or other host material) to break-possibly catastrophically.

The design of the rotary osteotome 22 enables it to simultaneously auto-graft and compact bone. The compaction aspect may be defined as the gentle push of osseous structure laterally outwardly to compact the cells throughout the region surrounding the osteotomy 20. The auto-grafting aspect is a result of some quantity of ground/milled bone that results from the apical tip of an osteotome 22 being forcibly advanced into a smaller osteotomy 20. At the point where the outermost edge of each rotating and forcibly advancing lip 34 contacts the bone, attrition causes the bone to be ground away. The bone debris collects mainly on the second trailing flanks 38, i.e., immediately behind the respective first trailing flanks 36. The auto-grafting phenomena supplements the basic bone compaction and condensation effects described above to further densify the inner walls of the osteotomy. Furthermore, auto-grafting—which is the process of repatriating the patient's own bone material—enhances natural healing properties in the human body to accelerate recovery and improve osscointegration.

To summarize, osseodensification is a method to preserve bone and its collagen content to enhance its plasticity. Osseodensification allows for enlarging an osteotomy 20 by compacting (and/or by cutting when rotation is reversed) with a high-speed rotary osteotomy 20, operated with a continuous supply of irrigating fluid, in preparation for a subsequently placed implant or anchoring fixture.

At least one valley 54 is fashioned in the apical end 32 of the osteotome 22. The valley 54 is a groove-like formation or relief in the tip, that passes between adjacent pairs of lips 34 and also cuts away the central portion of the lips 34. Preferably, the valley 54 will bisect, or generally bisect, the subtended angle between adjacent pairs of lips 34. Thus, in embodiments where the osteotome 22 has only two lips 34, as illustrated, a single valley 54 may extend generally perpendicular to lips 34. Said differently, in the two-lip embodiment show in the accompanying illustrations, the lips 34 are approximately 180-degrees apart which, when bisected by the valley 54, yields approximate right angles between the valley 54 and each lip 34. Nevertheless, it is contemplated that the valley 54 could unequally bisect the subtended angle between the adjacent pairs of lips 34, resulting in an acute angle on one side and an obtuse angle on the other.

In an exemplary embodiment where the osteotome is configured with four lips, the lips could be arranged approximately 90-degrees apart from one another, in which case the lips 34 would be considered equally circumferentially spaced apart from one another. Bisecting between two adjacent lips set 90-degrees apart from one another yields approximate 45-degree angles between the valley (or valleys) and each lip. Although it may be preferable that the valley 54 bisect, or generally bisect, the subtended angle between adjacent pairs of lips 34, such is not an absolute requirement so long as the valley 54 passes between adjacent pairs of lips 34. Moreover, in the contemplated four-lip embodiment the apical end of the osteotome could be fashioned with two valleys that cross one another at the longitudinal axis. In this example, each lip would be circumferentially offset from one of the valleys at approximately 45-degrees. But also possible is a four-lip embodiment provided with only one valley that bisects only two of the four adjacent lips. Such may become an attractive option in cases where the four lips are not equally circumferentially spaced apart from one another.

As previously mentioned, osteotomes 22 having an even number of lips (e.g., 2, 4, 6, etc.) are preferred over those having an odd number of lips (e.g., 3, 5, etc.). It will be appreciated that bisecting adjacent lips 34 with a valley 54 becomes problematic when the osteotome has an odd number of lips, necessitating shifting of the valley relative to the longitudinal axis or some other remedial action. Not so with osteotomes 22 having an even number of lips 34, and for this reason osteotomes 22 having two, four, etc. lips 34 are preferred so that the valley 54 may pass through the longitudinal axis without interfering with any of the other lips 34. Thus, while osteotomes 22 having an odd numbers of lips 34 are certainly possible and fall within the scope of the appended claims, there are perceived advantages for osteotomes 22 having an even number of lips 34 over those with an odd number of lips 34.

The valley 54 has upswept sidewalls 56 that truncate, or excise, the central portion of each lip 34. That is, the lip 34 is resected from the sidewall 56 inward by the valley 54. A floor 58 can be included between the sidewalls 56. The floor 58 may be generally flat, or gently curved, whereas the sidewalls 56 can have a moderate-to-steep profile. In the side elevation of FIG. 4, the floor 58 is shown in an embodiment to be generally perpendicular to the longitudinal axis of rotation with sidewalls 56 nearly perpendicular thereto.

The valley 54 is an element of, or at least a cooperative partner of, the debris control surface of the apical end 32. In most cases, the valley 54 will not intersect with the flutes 44. Therefore, in the illustrated examples, the axial depth of the valley 54 does not extend beyond the debris control surfaces (36-42) of the apical end 32. Or to say another way, the valley floor 58 is spaced (axially) from the plurality of flutes 44. Notwithstanding, it would not be outside the range of the person of ordinary skill to devise a valley 54 that exceeds the axial depth of the apical end 32 and thus enters into the area of the body 26 where the flutes 44 reside.

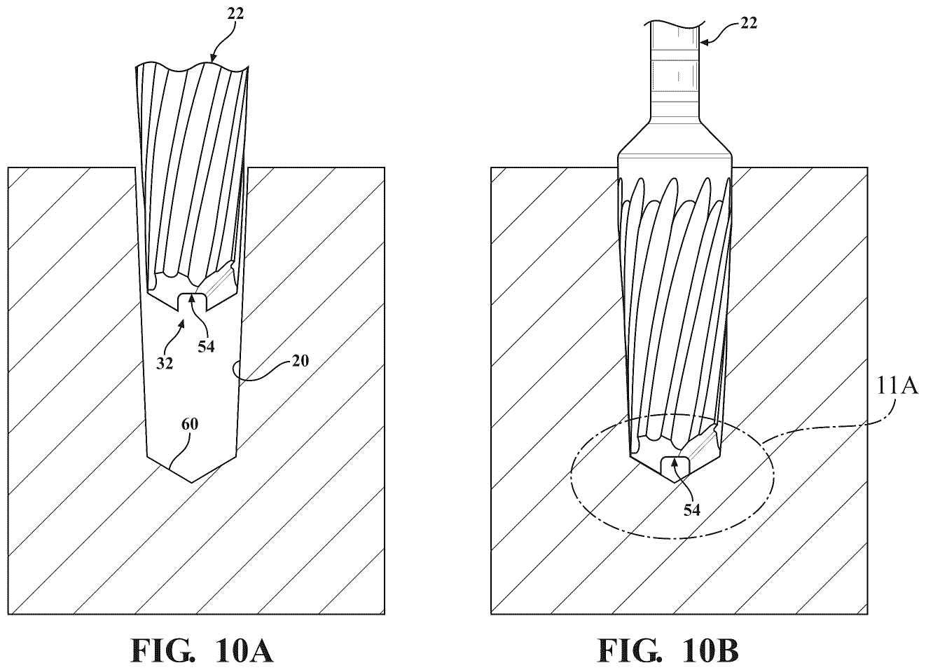

FIGS. 10A-10B offer a sequential illustration of the osteotomy enlargement process using the rotatory osteotome 22. FIG. 10A shows the rotary osteotome 22 entering the osteotomy 20 on descent toward its bottom 62. It is contemplated that the osteotome 22 will be driven by a surgical engine in either a non-cutting (i.e., densifying) direction or a cutting direction, depending on the bone conditions as assessed by the attending surgeon, similar to the depiction in FIG. 1. The rotational speed of the osteotome 22 will be greater than 200 rpm, typically between about 800-1500 rpm. FIG. 10B shows the rotary osteotome 22 reaching full depth in the osteotomy 20, having thus expanded the hole according to the geometry of the osteotome 22.

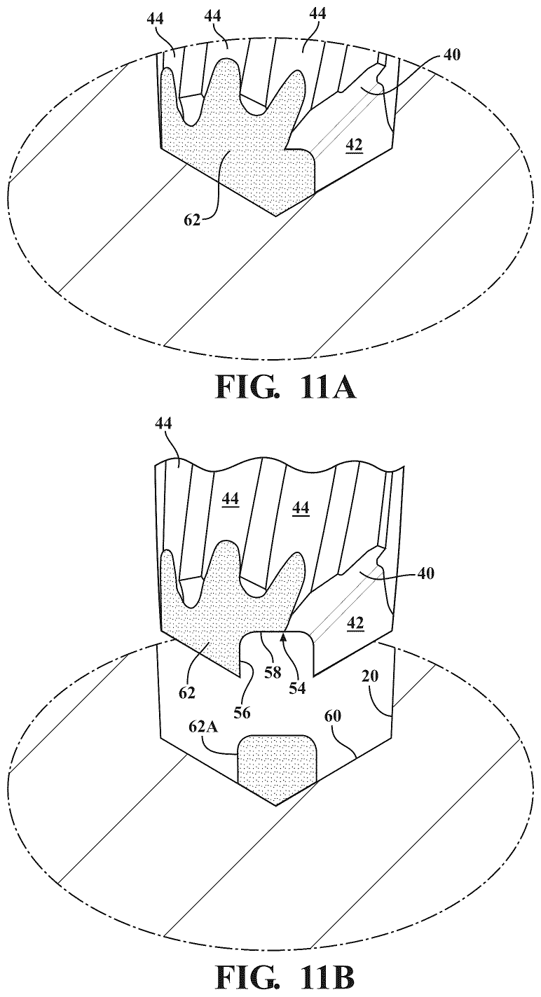

FIG. 11A is an enlarged view of the area circumscribed at 11A in FIG. 10B. A build-up of bone chips and bone debris 62 is shown in front of the second trailing flank 38, with migration into the flutes 44 where the boney particles 62 are re-patriated (i.e., auto-grafted) into the sidewalls of the osteotomy 20. It should be mentioned that both FIGS. 11A and 11B depict close-up views of the apical end 32 as having been rotated in a non-cutting direction where bone debris 62 can be seen piled-up against the second trailing flank 38. By contrast, rotation in a cutting direction would typically produce a pile-up of bone debris 62 against the lip face 42 and within the relief pocket 40.

FIG. 11B illustrates the subsequent withdrawal of the osteotome 22 from the osteotomy 20. From this view, it can be seen that a button-like formation 62A of bone chips 62 may be left behind at the center of the osteotomy bottom 62. The remnant button 62A is in the form of a small mound or nipple of drilling debris 62 that provides bone growth accelerant to facilitate new bone ingrowth and osseointegration of a subsequently installed anchor. Certain applications may find great benefit in the remnant button 62A. One such example could be the sinus lift procedure, in which the fully formed osteotomy 20 is typically packed with allograft prior to inserting the anchor. In such cases, the remnant button 62A serves as an activated bone growth accelerant effectively helping to seed the back-filled allograft material. Moreover, the small amount of bone chips 62 will be depressed, like a button, by the implant as it is screwed into position. If the implant has a domed or flattish leading end, the button 62A will be pressed in a localized fashion by the implant, thus helping to further gently stretch the sinus membrane. There is envisioned the creation of a novel anchor that is provided with a similar valley feature at its leading end. The remnant button 62A of bone fragments 62 will nest inside this complimentary valley in the anchor, creating a stability-enhancing feature. That is to say, the left-behind button 62A of bone fragments 62 may, when paired with a complimentary-configured anchor, provide over time an additional self-locking point or short tenon of bone that fills a mortise-like valley in the anchor to further resist lateral displacement under loading.

FIG. 12 is an end view of the rotary osteotome 22 according to an alternative embodiment in which the lands 50 are configured as margins for the working edge 52. FIG. 12 depicts exemplary migration patterns of bone debris into the longitudinal flutes 44. Despite being an alternative embodiment, the production and subsequent distribution of bone debris by the apical end 32 will be consistent with the previously described embodiment. In FIG. 12 it can be observed that the valley 54 has a beneficial effect on the flow of bone particles 62 from the surfaces 38, 40 of the apical end 32 into the flutes 44. In particular, the debris 62 is more equally distributed among the flutes 44, resulting in smoother operation of the osteotome 22 with less rotary imbalance. This can be especially beneficial in both cutting and densifying modes, where the additional cutting edges of the rotary osteotome 22 generates bone debris more rapidly than previous designs. The accumulated bone debris 62 pushed into the flutes 44 is carried away from the apical end 32, thereby reducing the possibility of heat-induced and/or pressure-induced damage to the bone particles 62.

FIGS. 5 and 12 are also useful to identify the enhanced distribution of cutting/grinding edges located at the apical end 32. The notch-like valley 54 forms several novel cut/grind edges at its intersections with the debris control surface. Specifically, in addition to the primary cut/grind edges formed by the lips 34, the osteotome 22 includes secondary cut/grind edges 64 along the interface of the valley 54 and the surfaces 36, 38, 40 and/or 42. Thus, by orienting the valley 54 so that it bisects adjacent lips 34, the number of cutting/grinding edges in the apical end 32 of a two-lipped osteotome 22 increases to six, as compared with typical prior art designs having only two. Embodiment of the osteotome 22 having more than two lips 34 will experience a proportionally greater increase in the number of cutting/grinding edges in the apical end 32. For example, a contemplated four-lip osteotome having two valleys 54 that crisscross over the longitudinal axis will increase to twelve, as compared with typical prior art designs having only four.

As a consequence, the combined lips 34 and secondary cut/grind edges 64 enable the rotary osteotome 22 to cut more aggressively when rotated in the cutting direction, and to grind more aggressively when rotated in the condensing direction. As mentioned already, a goal of the rotary osteotome 22 is to avoid removal of any bone debris 62 from the patient. However, the procedure of preparing the osteotomy 20 does necessarily generate some degree of bone debris 62. An advantage of having secondary cut/grind edges 64 is that the inevitable bone debris 62 will produced with less effort for the surgeon and with less trauma for the patient. Reduced trauma can also be beneficial to the health of the bone debris 62, and thereby facilitate autografting efficacy.

Bone debris 62 that is distributed into the flutes 44 works its way toward the lands 50 where, like a centrifuge, it is wiped and pressed into the sidewalls of the osteotomy 20 and there grafted back into the patient's body almost immediately after being dislocated. Bone debris 62 carried to the bottom 62 of the osteotomy 20 is wiped and pressed into the bottom of the osteotomy 20. Having produced the bone debris 62 with less trauma, its regenerative health is optimized. As a result, an auto-grafting zone is developed around and under the osteotomy 20, which fosters regrowth and long-term implant stability.

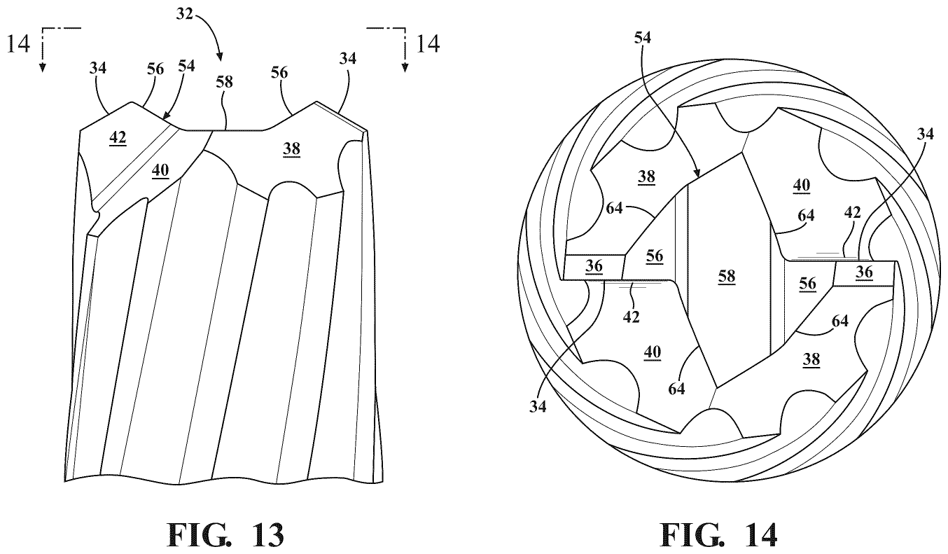

Other shapes for the valley 54 are contemplated, including a U-shaped cross-groove in which the floor 58 is semi-circular, a V-shaped cross-groove in which the floor 58 is merely the crevice where the sidewalls 56 intersect, as well as bowl-shaped profiles that are less valley-like and more crater-like, as well as other configurations. Another such alternative valley configuration is depicted in FIGS. 13 and 14, where the sidewalls 56 are splayed or tiled outwardly from the floor 58. The included angle between sidewalls 56 in this example is approximately 120-degrees (i.e., 60-degrees each side of the longitudinal axis). In this configuration, the lips 34 and first trailing flanks 36 are significantly shorted, which could be a disadvantage in certain applications and conditions. However, the remnant button (62A in FIG. 11B) will more easily release from the valley 54 in embodiment like this where the sidewalls 56 tilt outwardly akin to having a draft angle. In fact, angles ranging between very slight (e.g., 5 degrees) to very wide/broad (e.g. approaching 180-degrees) are possible.

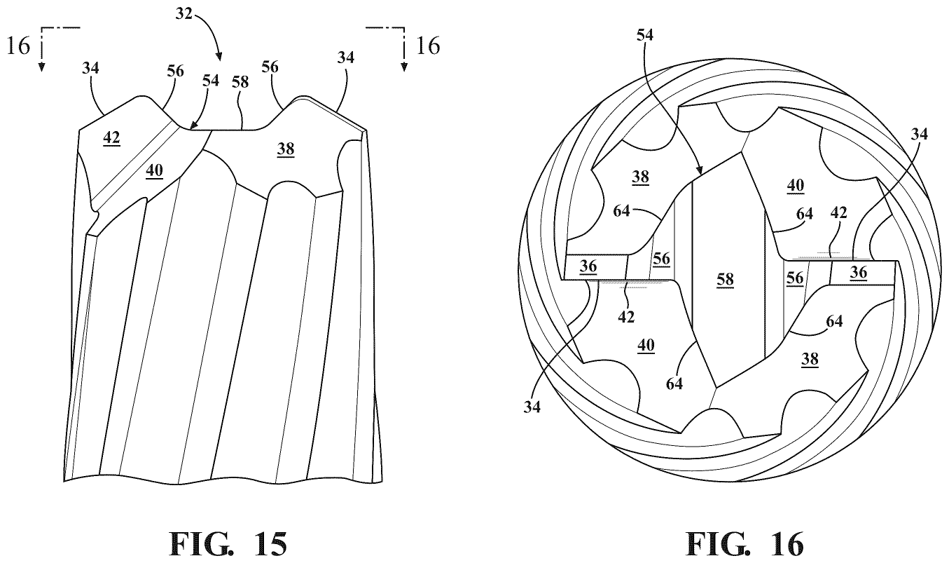

A still further alternative valley configuration is depicted in FIGS. 15 and 16, where the sidewalls 56 are again tiled outwardly from the floor 58. The included angle between sidewalls 56 in this example is approximately 90-degrees (i.e., 45-degrees each side of the longitudinal axis). To mention again, the included angle between the sidewalls 56 can range anywhere between 0 and something just less than 180-degrees. In this configuration, the corners created in the lips 34 and first trailing flanks 36 where they meet the sidewalls 56 are intentionally rounded or chamfered. The rounded corners could be an advantage in certain applications and conditions where sharpened leading corners are to be avoided. For an example, the embodiment of FIGS. 15 and 16 could be a good candidate for the sinus lift procedure. And as in all examples where there is a quasi-draft angle between the sidewalls 56, the remnant button (62A in FIG. 11B) will readily eject from the valley 54 upon withdrawal of the osteotome 22.

The foregoing invention has been described in accordance with the relevant legal standards, thus the description is exemplary rather than limiting in nature. Variations and modifications to the disclosed embodiment may become apparent to those skilled in the art and fall within the scope of the invention.

What is claimed is:

1. A rotary osteotome adapted for preparing an osteotomy with osseodensification effects, said rotary osteotome comprising:
a shank establishing a longitudinal axis of rotation, said shank having an upper end and a lower end,
a body extending axially from said lower end of said shank, said body having an apical end remote from said shank, a plurality of flutes disposed about said body,
each said flute having a working edge, said apical end having at least two lips, said at least two lips being arranged circumferentially about the longitudinal axis in at least one adjacent pair, a debris control surface extending between said adjacent pair of said lips, and
at least one valley passing though the longitudinal axis and between said adjacent pair of said lips, said valley intersecting at least a portion of said debris control surface, said valley establishing a plurality of secondary cut/grind edges at the intersections with said debris control surface.

2. The rotary osteotome of claim 1, wherein said valley generally bisects said adjacent pair of said lips.

3. The rotary osteotome of claim 1, wherein said valley includes upswept sidewalls truncating a central portion of said lips.

4. The rotary osteotome of claim 3, wherein said valley includes a floor disposed between said upswept sidewalls.

5. The rotary osteotome of claim 4, wherein said floor is axially spaced from said plurality of flutes.

6. The rotary osteotome of claim 4, wherein said floor is generally perpendicular to the longitudinal axis of rotation of said shank.

7. The rotary osteotome of claim 1, wherein said lips are non-diametrically offset from one another, and said debris control surface comprises a first trailing flank extending from each said lip in said adjacent pair.

8. The rotary osteotome of claim 7, wherein said debris control surface includes a second trailing flank extending from said first trailing flank.

9. The rotary osteotome of claim 8, wherein said debris control surface has a relief pocket extending from said second trailing flank.

10. The rotary osteotome of claim 1, wherein said debris control surface includes a lip face extending from said lip.

11. The rotary osteotome of claim 1, wherein said body has a conically tapered exterior profile decreasing from a maximum diameter adjacent said shank to a minimum diameter adjacent said apical end, each said flute spiraling helically about said body, each said working edge being substantially margin-less.

12. The rotary osteotome of claim 11, wherein each said working edge winds about said body in a direction that turns away from a non-cutting direction as said conically tapered profile decreases in diameter.

13. The rotary osteotome of claim 1, wherein each said flute has a cutting face on one side thereof defining a rake angle and a densifying face on the other side thereof defining a heel-side angle, a land formed between each adjacent pair of flutes.

14. A rotary osteotome adapted for preparing an osteotomy with osseodensification effects, said rotary osteotome comprising:
a shank establishing a longitudinal axis of rotation, said shank having an upper end and a lower end,
a body extending axially from said lower end of said shank, said body having an apical end remote from said shank, a plurality of flutes disposed about said body, each said flute having a working edge,
said apical end having at least two lips, said at least two lips being arranged circumferentially about the longitudinal axis in at least one adjacent pair, a debris control surface extending between said adjacent pair of said lips, said debris control surface comprising at least one trailing flank extending from one said lip in said adjacent pair, and
at least one valley passing though the longitudinal axis and bisecting said adjacent pair of said lips, said valley having upswept sidewalls truncating a central portion of said lips in said adjacent pair, said valley establishing a plurality of secondary cut/grind edges at the interface of said at least one trailing flank.

15. The rotary osteotome of claim 14, wherein said upswept sidewalls are outwardly angled from one another at a draft angle.

16. The rotary osteotome of claim 14, wherein said valley includes a floor disposed between said upswept sidewalls, said floor being axially spaced from said plurality of flutes.

17. The rotary osteotome of claim 14, further including a rounded or chamfered corner at the intersections of said valley with said lips.

18. The rotary osteotome of claim 16, wherein said floor is generally perpendicular to the longitudinal axis of rotation of said shank.

19. The rotary osteotome of claim 14, wherein said lips are non-diametrically offset from one another, and wherein said at least one trailing flank of said debris control surface comprises a first trailing flank extending from said lip.

20. A method for enlarging a precursor hole to receive a screw-in fixture, said method comprising the steps of:
rotating a rotary osteotome at greater than 200 RPM,
positioning the rotating rotary osteotome over a smaller precursor hole, the precursor hole having an interior surface extending between a generally circular entrance and a closed bottom, the interior surface of the precursor hole having a conically tapered sidewall,
enlarging the precursor hole by forcibly pushing the rotating body to the bottom of the precursor hole while concurrently autografting produced bone debris particles into the side walls of the enlarged hole, said enlarging step including bathing the rotary osteotome with irrigating fluid,
forming a button of bone debris at the bottom of the enlarged hole, and
withdrawing the rotary osteotome from the enlarged hole while the button of bone debris remains at the bottom of the enlarged hole.
